(19) United States
(12) Patent Application Publication
(10) Pub. No.: US 2006/0128726 A1
(43) Pub. Date: Jun. 15, 2006

(54) Process for preparing novel crystalline forms of (2S)-1-[[(7R)-7-(3,4-dichlorophenyl)-4,7-dihydro-5-methylpyrazolo[1,5]pyrimidine-6-yl]carbonyl]-2-(4-fluorophenyl)pyrrolidine, novel stable forms produced therein and formulations

(76) Inventors: Xuebao Wang, East Brunswick, NJ (US); Chiajen Lai, Kendall Park, NJ (US); Nachimuthu Soundararajan, Kendall Park, NJ (US); Nina Nguyen, North Brunswick, NJ (US); Weiming Ying, Martinsville, NJ (US); Lori S. Burton, Robbinsville, NJ (US); Rajesh Babulal Gandhi, Plainsboro, NJ (US); Krishnaswamy Srinivas Raghavan, Cranbury, NJ (US); Ronald C. West, Lawrenceville, NJ (US); Yuji Simon Zhou, Freehold, NJ (US); Lin Yan, East Brunswick, NJ (US); Xiaotian Yin, Plainsboro, NJ (US); John D. DiMarco, East Brunswick, NJ (US); Jack Z. Gougoutas, Princeton, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/283,651

(22) Filed: Nov. 21, 2005

Related U.S. Application Data
(60) Provisional application No. 60/630,593, filed on Nov. 24, 2004.

(51) Int. Cl. A61K 31/519; C07D 487/04

(52) U.S. Cl. 514/259.3; 544/281

(57) ABSTRACT

A process is provided for preparing novel crystalline forms, namely selectively and consistently preparing the H2-1 dihydrate crystalline form, the H2-2 dihydrate crystalline form, the N-3 anhydrate crystalline form and the MTBE solvate crystalline form of the IKur compound (2S)-1-[[(7R)-7-(3,4-dichlorophenyl)-4,7-dihydro-5-methylpyrazolo[1,5]pyrimidine-6-yl]carbonyl]-2-(4-fluorophenyl)pyrrolidine. The process includes preparation of the H2-1 and H2-2 forms which are used in preparing the N-3 anhydrate form which is particularly stable and has suitable flow properties and desired particle size. Novel H2-1 dihydrate and H2-2 dihydrate forms, the N-3 anhydrate form and the MTBE solvate form of the above IKur compound, pharmaceutical compositions containing such novel forms and a method for preventing or treating arrhythmias including atrial fibrillation and IKur related conditions employing such novel forms are also provided.

This application claims a benefit of priority from U.S. Provisional Application No. 60/630,593, filed Nov. 24, 2004, the entire disclosure of which is herein incorporated by reference.

### FIELD OF THE INVENTION

The present invention relates to a process for preparing novel stable crystalline forms, including the H2-1 dihydrate, H2-2 dihydrate, the MTBE solvate and N-3 anhydrate crystalline forms, of the IKur compound (2S)-1-[[(7R)-7-(3,4-dichlorophenyl)-4,7-dihydro-5-methylpyrazolo[1,5]pyrimidine-6-yl]carbonyl]-2-(4-fluorophenyl)pyrrolidine, to such novel H2-1 and H2-2 dihydrate forms, the corresponding MTBE solvate and the N-3 anhydrate forms, to pharmaceutical compositions containing such novel crystalline forms, and to methods of treating a mammal suffering from a cardiac arrhythmia such as atrial fibrillation and related conditions employing such novel crystalline forms.

### BACKGROUND OF THE INVENTION

U.S. Pat. No. 6,706,720 to Atwal et al. discloses heterocyclic dihydropyridimine compounds which are useful as inhibitors of potassium channel function, especially inhibitors of the Kᵥ1 subfamily of voltage-gated K⁺ channels, and more especially inhibitors of Kᵥ1.5 which have been linked to the atrial-specific, ultrarapid delayed rectifier potassium current (IKur) for the prevention/termination of arrhythmias such as atrial fibrillation and other IKur-associated disorders. One such compound disclosed in the '720 Atwal patent is (2S)-1-[[(7R)-(3,4-dichlorophenyl)-4,7-dihydro-5-methylpyrazolo[1,5-]pyrimidine-6-yl]carbonyl]-2-(4-fluorophenyl)pyrrolidine which has the structure 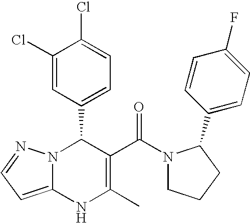
and is a selective blocker of the ultrarapidly activating, sustained potassium current (IKur, also known as Kᵥ1.5) (and is hereinafter referred to as “the IKur compound” or “free base”). The aqueous solubility of the IKur compound is low (2-4 μg/mL depending on crystalline form).

### BRIEF DESCRIPTION OF THE INVENTION

In accordance with the present invention, a process is provided for selectively preparing novel crystalline dihydrate, anhydrate and solvate forms of the IKur compound which is the free base of the structure I: 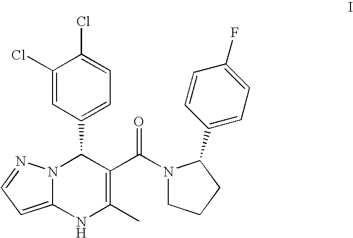
including the H2-1 dihydrate, H2-2 dihydrate, N-3 anhydrate and methyl t-butyl ether (MTBE) solvate forms.

It has been found that the free base of structure I in its amorphous form and crystalline forms are sensitive to light and heat.

The N-3 anhydrate form of the free base I which is the preferred crystalline form, is a physically stable, neat crystalline form which has good crystallinity, acceptable thermal properties, low hygroscopicity, satisfactory solid-state stability (physical and chemical), and satisfactory oral bioavailability, and can be prepared having a controlled desired particle size (D90≦50 μμm).

The H2-1 dihydrate (triclinic) of the free base I and the H2-2 dihydrate (monoclinic) of the free base I crystallize readily from aqueous/organic solvent systems. However, both the H2-1 and H2-2 crystalline forms undergo dehydration and transform to amorphous solids at about 40° to 50° C. Mixtures of H2-1 and H2-2 forms in aqueous ethanol slurries convert completely to the H2-2 dihydrate, which accordingly is thermodynamically more stable than the H2-1 dihydrate. Inasmuch as conversion of H2-1 form to H2-2 form has been observed at 5° C., 25° C., 50° C. and 80° C., the polymorphs are monotropically related with the H2-2 form being the more stable form at all temperatures.

Stable neat N-3 anhydrate form, mp˜210° C., crystallizes from the H2-2 form in absolute ethanol.

The MTBE solvate of the free base (hereinafter the MTBE solvate) may be obtained by slurrying the amorphous free base compound I in MTBE. The MTBE solvate has fair thermal stability and does not de-solvate until>100° C.

In accordance with the present invention, a process is provided for preparing the H2-1 dihydrate crystalline form of the IKur compound which includes the steps of:

a) providing the IKur compound, such as in the form of an amorphous solid, H2-1 dihydrate or H2-2 dihydrate or mixtures of two or more thereof, and which in one embodiment may be optionally in admixture with n-butyl alcohol, and concentrated hydrochloric acid, at a reduced temperature below about 2.5° C.;

b) mixing the IKur compound from step a) with an alkanol, preferably ethyl alcohol, more preferably absolute ethyl alcohol, at a reduced temperature, within the range from about −10 to about 10° C., preferably from about 0 to about 5° C.; and

c) treating the reaction mixture from step b) with a strong base such as an alkali metal hydroxide, preferably sodium hydroxide while maintaining the reaction mixture at a reduced temperature within the range from about 10 to about −25° C., preferably from about 5 to about −15° C., more preferably from about 0 to about −2.5° C., and optionally adding trisodium phosphate Na₃PO₄ to adjust pH of the reaction mixture within the range from about 6.5 to about 9, preferably from about 8 to about 9, to cause precipitation of the H2-1 dihydrate.

In accordance with the present invention, a process is provided for preparing the H2-2 dihydrate crystalline form of the IKur compound I which includes the steps of:

a) seeding the reaction slurry of the H2-1 dihydrate of the IKur compound (as described in step c) above) or a slurry of H2-1 dihydrate in a mixture of 1-butanol, ethanol and sodium phosphate buffer or ethanol, with crystals of H2-2 dihydrate;

b) adjusting pH of the reaction mixture from step a) within the range from about 5.5 to about 8.5, preferably from about 6 to about 7.5, for example, by mixing the reaction mixture with saturated trisodium phosphate; and

c) heating the reaction mixture from step b) at a temperature within the range from about 30 to about 80° C., preferably from about 35 to about 45° C., to form the H2-2 crystalline dihydrate.

In accordance with the present invention, a process is provided for preparing the anhydrate crystalline N-3 form of the IKur compound I which includes the steps of:

a) providing the dihydrate crystalline H2-2 form of the IKur compound;

b) treating the dihydrate crystalline H2-2 form with an alkanol, preferably ethyl alcohol, more preferably absolute ethyl alcohol;

c) optionally seeding the reaction mixture from step b) with crystals of N-3 anhydrate of the IKur compound to form a slurry;

d) heating the slurry from step c) at an elevated temperature, within the range from about 35 to about 50° C., preferably from about 40 to about 45° C., to cause formation of crystals of the N-3 anhydrate; and

e) drying the resulting reaction product to recover crystals of the N-3 anhydrate.

In another embodiment of the invention, a process is provided for preparing the crystalline methyl t-butyl ether (MTBE) solvate form of the IKur compound which includes the steps of:

a) forming a slurry of the IKur compound, in the form of an amorphous solid, H2-1 dihydrate, H2-2 dihydrate or a mixture of two or more thereof, in MTBE; and

b) mixing the slurry from step a) with seeds of MTBE solvate to form MTBE solvate.

In addition, in accordance with the present invention, a process is provided for preparing the crystalline H2-2 dihydrate form of the IKur compound by recrystallization from the IKur compound, which includes the steps of:

a) dissolving the IKur compound (which may be amorphous, H2-1 dihydrate and/or H2-2 dihydrate) in ethyl alcohol;

b) adding the solution of IKur compound to an aqueous slurry of seeds of crystalline H2-2 dihydrate form of the IKur compound to form a slurry;

c) heating the slurry from step b) at a temperature within the range from about 35° C. to about 65° C., preferably from about 45° C. to about 55° C.;

d) optionally adding ethyl alcohol to the reaction mixture from step c);

e) optionally adding additional seeds of crystalline H2-2 dihydrate form to the reaction mixture from step c) or step d), to form H2-2 dihydrate; and

f) optionally cooling the reaction mixture from step e) at a temperature within the range from about −5 to about 10° C., preferably from about 0 to about 5° C., and recovering H2-2 dihydrate.

In accordance with the present invention, the H2-1 dihydrate crystalline form of the IKur compound is novel and is identified by the observed and simulated powder x-ray diffraction (PXRD) patterns shown in FIGS. 1 and 7 or in the observed PXRD pattern, by the first 5 major peaks; the single crystal x-ray diffraction data and crystallographic data shown in Example 1; and by the differential scanning calorimetry (DSC) and thermogravimetric analysis (TGA) thermograms shown in FIGS. 2 and 2A, respectively.

In accordance with the present invention, the H2-2 dihydrate crystalline form of the IKur compound is novel and is identified by the observed and simulated powder x-ray diffraction (PXRD) patterns shown in FIGS. 3 and 7 or in the observed PXRD pattern, by the first 5 major peaks; the single crystal x-ray diffraction data and crystallographic data shown in Examples 1, 4 and 5; and by the differential scanning calorimetry and thermogravimetric analysis thermograms shown in FIGS. 4 and 4A.

In accordance with the present invention, the N-3 anhydrate crystalline form of the IKur compound is novel and is identified by the observed and simulated powder x-ray diffraction (PXRD) patterns shown in FIGS. 5, 7 and 7A or in the observed PXRD pattern, by the first 5 major peaks; the single crystal x-ray diffraction data and crystallographic data shown in Example 2; and by the differential scanning calorimetry and thermogravimetric analysis thermograms shown in FIGS. 6 and 6A, respectively.

In accordance with the present invention, the MTBE solvate crystalline form of the IKur compound is novel and is identified by the observed powder and variable temperature (VT) powder x-ray diffraction (PXRD) patterns shown in FIGS. 8 and 8A, respectively; and by the differential scanning calorimetry and thermogravimetric analysis thermograms shown in FIGS. 9 and 9A, respectively.

In addition, in accordance with the present invention, a novel pharmaceutical composition is provided which includes the IKur compound in amorphous form or in crystalline form such as the crystalline N-3 anhydrate, H2-1 dihydrate, H2-2 dihydrate, or MTBE solvate, preferably the N-3 anhydrate, and a pharmaceutically acceptable vehicle therefor. In a preferred embodiment, the N-3 anhydrate composition will be in the form of an oral solution or suspension. Where the composition is in the form of an oral solution, it will include a solubilizer such as d-alpha tocopheryl polyethylene glycol 1000 succinate (TPGS), a consistency enhancer and solubilizer such as ethanol, a surfactant and solubilizer such as polyoxyethylene (20) sorbitan monooleate (Tween 80), and a base vehicle and solubilizer such as polyethylene glycol 400 (PEG 400, MW400, viscosity (210° F.) 7.3 centistokes.

Where the composition is in the form of an oral suspension, it will preferably include the N-3 anhydrate, a surfactant such as Tween 80, and purified water. However, the oral suspension may include the other solid forms of the IKur compound including the amorphous form, the H2-1 dihydrate, the H2-2 dihydrate and the MTBE solvate.

### BRIEF DESCRIPTION OF THE FIGURES

FIG. 1 depicts observed and simulated powder x-ray diffraction patterns of the crystalline H2-1 dihydrate form of (2S)-1-[[(7R)-7-(3,4-dichlorophenyl)-4,7-dihydro-5-methylpyrazolo[1,5]pyrimidine-6-yl]carbonyl]-2-(4-fluorophenyl)pyrrolidine;

FIGS. 2 and 2A depict differential scanning calorimetry and thermogravimetric analysis thermograms of the H2-1 dihydrate crystalline form of (2S)-1-[[(7R)-7-(3,4-dichlorophenyl)-4,7-dihydro-5-methylpyrazolo[1,5]pyrimidine-6-yl]carbonyl]-2-(4-fluorophenyl)pyrrolidine;

FIG. 3 depicts observed and simulated powder x-ray diffraction patterns of the crystalline H2-2 dihydrate form of (2S)-1-[[(7R)-7-(3,4-dichlorophenyl)-4,7-dihydro-5-methylpyrazolo[1,5]pyrimidine-6-yl]carbonyl]-2-(4-fluorophenyl)pyrrolidine;

FIGS. 4 and 4A depict differential scanning calorimetry and thermogravimetric analysis thermograms of the crystalline H2-2 dihydrate form of (2S)-1-[[(7R)-7-(3,4-dichlorophenyl)-4,7-dihydro-5-methylpyrazolo[1,5]pyrimidine-6-yl]carbonyl]-2-(4-fluorophenyl)pyrrolidine;

FIG. 5 depicts observed and simulated powder x-ray diffraction pattern of the crystalline N-3 anhydrate form of (2S)-1-[[(7R)-7-(3,4-dichlorophenyl)-4,7-dihydro-5-methylpyrazolo[1,5]pyrimidine-6-yl]carbonyl]-2-(4-fluorophenyl)pyrrolidine;

FIGS. 6 and 6A depict differential scanning calorimetry and thermogravimetric analysis thermograms of the crystalline N-3 anhydrate form of (2S)-1-[[(7R)-7-(3,4-dichlorophenyl)-4,7-dihydro-5-methylpyrazolo[1,5]pyrimidine-6-yl]carbonyl]-2-(4-fluorophenyl)pyrrolidine;

FIG. 7 depicts combined simulated powder x-ray diffraction patterns from FIGS. 1, 3 and 5;

FIG. 7A depicts the simulated powder x-ray diffraction pattern for N-3 form from FIGS. 1 and 7;

FIGS. 8 and 8A depict powder and VT x-ray diffraction patterns of the crystalline MTBE solvate form of (2S)-1-[[(7R)-7-(3,4-dichlorophenyl)-4,7-dihydro-5-methylpyrazolo[1,5]pyrimidine-6-yl]carbonyl]-2-(4-fluorophenyl)pyrrolidine; and

FIGS. 9 and 9A depict differential scanning calorimetry and thermogravimetric analysis thermograms, respectively, of the crystalline MTBE solvate form of (2S)-1-[[(7R)-7-(3,4-dichlorophenyl)-4,7-dihydro-5-methylpyrazolo[1,5]pyrimidine-6-yl]carbonyl]-2-(4-fluorophenyl)pyrrolidine.

### DETAILED DESCRIPTION OF THE INVENTION

The terms N-3 anhydrate, neat N-3 form and the N-3 neat form are used interchangeably and refer to the N-3 form which is free of water or any other solvent in the crystal structure.

In carrying out the process for forming the crystalline N-3 anhydrate, the reaction mixture formed of the crystalline H2-2 dihydrate form of the IKur compound, the alcohol, preferably ethyl alcohol, and the seeds of crystals of the N-3 anhydrate form of the IKur compound, are heated at a temperature within the range from about 35 to about 50° C., preferably from about 40 to about 45° C., for a period within the range from about 0.5 to about 20 hours, preferably from about 2 to about 15 hours, more preferably from about 5 to about 12 hours, and then maintained at ambient temperature for a period from about 0.5 hours to about 3 days, preferably from about 18 hours to about 56 hours, under an inert atmosphere such as a nitrogen atmosphere.

The ethyl alcohol will be employed in a molar ratio to the crystalline H2-2 dihydrate form within the range from about 14:1 to about 42:1, preferably from about 23:1 to about 35:1, and the seeds of the crystalline N-3 anhydrate form are employed in a molar ratio to the crystalline H2-2 dihydrate form within the range from about 0.001:1 to about 0.020:1, preferably from about 0.005:1 to about 0.015:1.

Alcohols other than ethyl alcohol which may be employed in forming the anhydrate crystalline N-3 form include, but are not limited to methanol, 2-propanol and 1-butanol.

In carrying out the process for preparing dihydrate forms H2-1 and H2-2, where employed, the n-butyl alcohol, will be employed in a molar ratio to the starting IKur compound within the range from about 12:1 to about 24:1, preferably from about 14:1 to about 22:1, and the concentrated hydrochloric acid (from about 10 to about 12 N HCl), will be employed in a molar ratio to the starting IKur compound within the range from about 4:1 to about 10:1, preferably from about 5.5:1 to about 7.5:1.

The reaction mixture from step a) above or the IKur compound such as in amorphous form, will preferably be treated with ethyl alcohol employing a molar ratio of ethyl alcohol to IKur compound within the range from about 30:1 to about 60:1, preferably from about 35:1 to about 55:1.

The reaction mixture from step b) or step c) will be treated with a strong base such as an alkali hydroxide, such as KOH, NaOH or LiOH, preferably NaOH, and optionally trisodium phosphate, to adjust the pH of the reaction mixture within the range from about 6.5 to about 9, preferably from about 8 to about 9.

In forming the H2-2 dihydrate, the reaction slurry containing precipitated H2-1 dihydrate (from the H2-1 dihydrate preparation), or H2-1 dihydrate slurried in a mixture of 1-butanol, ethanol and sodium phosphate buffer, or ethanol, is seeded with crystals of H2-2 dihydrate employing a molar ratio of H2-2 dihydrate crystals: H2-1 dihydrate within the range from about 0.001:1 to about 0.020:1, preferably from about 0.005:1 to about 0.015:1. The pH of the H2-2 dihydrate seeded reaction slurry is adjusted to within the range from about 5.5 to about 8.5, preferably from about 6 to about 7.5, more preferably about 7.5 with a strong acid such as hydrochloric acid and the reaction mixture is heated at a temperature within the range from about 30 to about 80° C., preferably from about 35 to about 45° C., more preferably about 40° C.

In carrying out the process of the invention for preparing the MTBE solvate, the MTBE will be employed in a molar ratio to the H2-1 dihydrate within the range from about 30:1 to about 60:1, preferably from about 40:1 to about 50:1.

The seeds of MTBE solvate will be employed in a molar ratio to the starting H2-1 dihydrate within the range from about 0.001:1 to about 0.020:1, preferably from about 0.005:1 to about 0.015:1.

In carrying out the process of the invention for preparing the H2-2 dihydrate by recrystallization from the IKur compound in the form of a) an amorphous solid or b) H2-1 dihydrate or c) H2-2 dihydrate and/or a mixture of a) and b), b) and c), a) and c) or a), b) and c) (without requiring the presence of n-butyl alcohol and HCl), the IKur compound will be dissolved in ethyl alcohol employing a molar ratio of ethyl alcohol to the IKur compound within the range from about 30:1 to about 50:1, preferably from about 35:1 to about 45:1.

The aqueous slurry of seeds of H2-2 dihydrate will be added to the solution of IKur compound in ethyl alcohol (and not the reverse) and the seeds of H2-2 dihydrate will be employed in a molar ratio to the IKur compound within the range from about 0.001:1 to about 0.010:1, preferably from about 0.002:1 to about 0.005:1. Additional ethyl alcohol will be optionally added to the heated slurry containing previously added seeds of H2-2 dihydrate in a molar ratio of ethyl alcohol to the IKur compound within the range from about 10:1 to about 30:1, preferably from about 15:1 to about 25:1. The optional additional seeds will be added in a molar ratio to the previously added H2-2 dihydrate seeds within the range from about 0.001:1 to about 0.015:1, preferably from about 0.004:1 to about 0.008:1.

The H2-1 dihydrate, H2-2 dihydrate, N-3 anhydrate and MTBE solvate of the present invention (referred to as the compounds of the present invention) inhibit the Kᵥ1 subfamily of voltage-gated K⁺ channels, and as such are useful in the treatment and/or prevention of various disorders: cardiac arrhythmias, including supraventricular arrhythmias, atrial arrhythmias, atrial flutter, atrial fibrillation, complications of cardiac ischemia, and use as heart rate control agents; angina pectoris including relief of Prinzmetal's symptoms, vasospastic symptoms and variant symptoms; gastrointestinal disorders including reflux esauphagitis, functional dyspepsia, motility disorders (including constipation and diarrhea), and irritable bowel syndrome; disorders of vascular and visceral smooth muscle including asthma, chronic obstructive pulmonary disease, adult respiratory distress syndrome, peripheral vascular disease (including intermittent claudication), venous insufficiency, impotence, cerebral and coronary spasm and Raynaud's disease; inflammatory and immunological disease including inflammatory bowel disease, rheumatoid arthritis, graft rejection, asthma chronic obstructive pulmonary disease, cystic fibrosis and atherosclerosis; cell proliferative disorders including restenosis and cancer (including leukemia); disorders of the auditory system; disorders of the visual system including macular degeneration and cataracts; diabetes including diabetic retinopathy, diabetic nephropathy and diabetic neuropathy; muscle disease including myotonia and wasting; peripheral neuropathy; cognitive disorders; migraine; memory loss including Alzheimer's and dementia; CNS mediated motor dysfunction including Parkinson's disease, and ataxia; epilepsy; and other ion channel mediated disorders.

As inhibitors of the Kᵥ1 subfamily of voltage-gated K⁺ channels the above compounds of the present invention are useful to treat a variety of disorders including resistance by transplantation of organs or tissue, graft-versus-host diseases brought about by medulla ossium transplantation, rheumatoid arthritis, systemic lupus erythematosus, hashimoto's thyroiditis, multiple sclerosis, myasthenia gravis, type I diabetes uveitis, juvenile-onset or recent-onset diabetes mellitus, posterior uveitis, allergic encephalomyelitis, glomerulonephritis, infectious diseases caused by pathogenicmicroorganisms, inflammatory and hyperproliferative skin diseases, psoriasis, atopical dermatitis, contact dermatitis, eczematous dermatitises, seborrhoeisdermatitis, Lichen planus, Pemphigus, bullous pemphigoid, Epidermolysis bullosa, urticaria, angioedemas, vasculitides, erythemas, cutaneous eosinophilias, Lupus erythematosus, acne, Alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer Scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns and leukotriene B₄-mediated diseases, Coeliaz diseases, proctitis, eosinophilic gastroenteritis, mastocytosis, Crohn's disease, ulcerative colitis, migraine, rhinitis, eczema, interstitial nephritis, Good-pasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia osses dentis, glomerulonephritis, male pattern alopecia or alopecia senilis by preventing epilation or providing hair germination and/or promoting hair generation and hair growth, muscular dystrophy; Pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis, pigentosa, senile macular degeneration, vitreal scarring, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenis, metastatis of carcinoma and hypobaropathy, disease caused by histamine or leukotriene-C₄ release, Behcet's disease, autoimmune hepatitis, primary biliary cirrhosis sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augention of chemotherapeutic effect, cytomegalovirus infection, HCMV infection, AIDS, cancer, senile dementia, trauma, and chronic bacterial infection.

The compounds of the present invention are antiarrhythmic agents which are useful in the prevention and treatment (including partial alleviation or cure) of arrhythmias. As inhibitors of Kᵥ1.5 compounds within the scope of the present invention are particularly useful in the selective prevention and treatment of supraventricular arrhythmias such as atrial fibrillation, and atrial flutter. By “selective prevention and treatment of supraventricular arrhythmias” is meant the prevention or treatment of supraventricular arrhythmias wherein the ratio of the prolongation of the atrial effective refractory period to the prolongation of the ventricular effective refractory period is greater than 1:1. This ratio is preferably greater than 4:1, more preferably greater than 10:1, and most preferably such that prolongation of the atrial effective refractory response period is achieved without significantly detectable prolongation of the ventricular effective refractory period.

In addition, the compounds within the scope of the present invention block IKur, and thus may be useful in the prevention and treatment of all IKur-associated conditions. An “IKur-associated condition” is a disorder which may be prevented, partially alleviated or cured by the administration of an IKur blocker. The Kᵥ1.5 gene is known to be expressed in stomach tissue, intestinal/colon tissue, the pulmonary artery, and pancreatic beta cells. Thus, administration of an IKur blocker could provide useful treatment for disorders such as: reflux esauphagitis, functional dispepsia, constipation, asthma, and diabetes. Additionally, Kᵥ1.5 is known to be expressed in the anterior pituitary. Thus, administration of an IKur blocker could stimulate growth hormone secretion. IKur inhibitors can additionally be useful in cell proliferative disorders such as leukemia, and autoimmune diseases such as rheumatoid arthritis and transplant rejection.

The present invention thus provides methods for the prevention or treatment of one or more of the aforementioned disorders employing compounds of the present invention. Other therapeutic agents such as those described below may be employed with the inventive compounds in the present methods. In the methods of the present invention, such other therapeutic agent(s) may be administered prior to, simultaneously with or following the administration of the compound(s) of the present invention.

The present invention also provides pharmaceutical compositions which include at least one of the compounds of the present invention capable of preventing or treating one or more of the aforementioned disorders in an amount effective therefor, and a pharmaceutically acceptable vehicle or diluent. The compositions of the present invention may contain other therapeutic agents as described below, and may be formulated, for example, by employing conventional solid or liquid vehicles or diluents, as well as pharmaceutical additives of a type appropriate to the mode of desired administration (for example, excipients, such as fillers, binders, preservatives, stabilizers, solubilizers, flavors, etc.) according to techniques such as those well known in the art of pharmaceutical formulation.

The compounds of the present invention may be administered by any suitable means, for example, orally, such as in the form of tablets, capsules, granules, powders, aqueous and non-aqueous oral solutions and suspensions; sublingually; bucally; parenterally, such as by subcutaneous, intravenous, intramuscular, or intrastemal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The compounds of the present invention may, for example, be administered in a form suitable for immediate release or extended release. Immediate release or extended release may be achieved by the use of suitable pharmaceutical compositions containing the compounds of the present invention, or, particularly in the case of extended release, by the use of devices such as subcutaneous implants or osmotic pumps. In the case where the compounds of the present invention are being administered to prevent or treat arrhythmias, the compounds may be administered to achieve chemical conversion to normal sinus rhythm, or may optionally be used in conjunction with electrical cardioconversion.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The compounds of formula I may also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

The oral composition of the invention in the form of an oral solution will include as excipients:

a) a solubilizer, preferably d-alpha tocopheryl polyethylene glycol 1000 succinate (TPGS), in an amount within the range from about 15 to about 20%, preferably about 20% by weight/weight of the composition;

b) a surfactant, preferably polyoxyethylene (20) sorbitan monooleate, in an amount within the range from about 7 to about 10%, preferably about 8.5% by weight/weight of the composition;

c) a consistency enhancer and solubilizer, preferably ethanol, in an amount within the range from about 8 to about 12%, preferably about 10% by weight/weight of the composition; and

d) base vehicle and solubilizer, preferably polyethylene glycol 400, in an amount within the range from about 50 to about 70%, preferably about 61.5% by weight/weight of the composition.

In a preferred embodiment, the oral solution will contain from about 5 to about 30 mg/mL, preferably from about 5 to about 20 mg/mL of the N-3 anhydrate. However, it will be appreciated that the oral solution may contain the IKur compound in any other form such as the amorphous solid, H2-1 dihydrate, H2-2 dihydrate and MTBE solvate.

The oral suspension for the N-3 anhydrate of the invention will include as excipients:

a) a surfactant, preferably polyoxyethylene (20) sorbitan monooleate, in an amount within the range from about 0.01 to about 0.1%, preferably about 0.05% by weight/weight of the composition; and

b) purified water in an amount within the range from about 99 to about 99.98, preferably about 99.95% by weight/weight of the composition.

The oral suspension will preferably contain from about 2 to about 10 mg/mL, preferably about 2 mg/mL of the N-3 anhydrate. However, it will be appreciated that the oral suspension may contain the IKur compound in any other form such as amorphous form, H2-1 dihydrate, H2-2 dihydrate and MTBE solvate.

The term ethanol or ethyl alcohol as employed herein includes absolute ethanol and mixtures of ethanol and water up to about 5% by volume.

Exemplary compositions for nasal aerosol or inhalation administration include solutions in saline which may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, a suitable non-irritating excipient, such as cocoa butter, synthetic glyceride esters or polyethylene glycols, which are solid at ordinary temperatures, but liquefy and/or dissolve in the rectal cavity to release the drug.

Exemplary compositions for topical administration include a topical carrier such as Plastibase (mineral oil gelled with polyethylene).

The effective amount of a compound of the present invention may be determined by one of ordinary skill in the art, and includes exemplary dosage amounts for an adult human of from about 0.001 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to the aforementioned disorders.

The compounds of the present invention may be employed alone or in combination with each other and/or other suitable therapeutic agents useful in the treatment of the aforementioned disorders or other disorders, including: other antiarrhythmic agents such as Class I agents (e.g., propafenone), Class II agents (e.g., carvadiol and propranolol), Class III agents (e.g., sotalol, dofetilide, amiodarone, azimilide and ibutilide), Class IV agents (e.g., diltiazem and verapamil), 5HT antagonists (e.g., sulamserod, serraline and tropsetron), and dronedarone; calcium channel blockers (both L-type and T-type) such as diltiazem, verapamil, nifedipine, amlodipine and mybefradil; Cyclooxygenase inhibitors (i.e., COX-1 and/or COX-2 inhibitors) such as aspirin, indomethacin, ibuprofen, piroxicam, naproxen, Celebrex®, Vioxx® and NSAIDs; anti-platelet agents such as GPIIb/IIIa blockers (e.g., abciximab, eptifibatide and tirofiban), P2Y₁₂ antagonists (e.g., clopidogrel, ticlopidine and CS-747), thromboxane receptor antagonists (e.g., ifetroban), aspirin, and PDE-III inhibitors (e.g., dipyridamole) with or without aspirin; diruetics such as chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, and spironolactone; anti-hypertensive agents such as alpha adrenergic blockers, beta adrenergic blockers, calcium channel blockers, diuretics, renin inhibitors, ACE inhibitors, (e.g., captropril, zofenopril, fosinopril, enalapril, ceranopril, cilazopril, delapril, pentopril, quinapril, ramipril, lisinopril), A II antagonists (e.g., losartan, irbesartan, valsartan), ET antagonists (e.g. sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), nitrates, and combinations of such anti-hypertensive agents; antithrombotic/thrombolytic agents such as tissue plasminogen activator (tPA), recombinant tPA, tenecteplase (TNK), lanoteplase (nPA), factor VIIa inhibitors, factor Xa inhibitors, thromin inibitors (e.g., hirudin and argatroban), PAI-1 inhibitors (i.e., inactivators of tissue plasminogen activator inhibitors), α2-antiplasmin inhibitors, streptokinase, urokinase, prourokinase, anisoylated plasminogen streptokinase activator complex, and animal or salivary gland plasminogen activators; anticoagulants such as warfarin and heparins (including unfractionated and low molecular weight heparins such as enoxaparin and dalteparin); HMG-CoA reductase inhibitors such as pravastatin, lovastatin, atorvastatin, simvastatin, NK-104 (a.k.a. itavastatin, or nisvastatin or nisbastatin) and ZD-4522 (a.k.a. rosuvastatin, or atavastatin or visastatin); other cholesterol/lipid lowering agents such as squalene synthetase inhibitors, fibrates, and bile acid sequestrants (e.g., questran); antipoliferative agents such as cyclosporin A, taxol, FK 506, and adriamycin; antitumor agents such as taxol, adriamycin, epothilones, cisplatin and carboplatin; anti-diabetic agents such as biguanides (e.g. metformin), glucosidase inhibitors (e.g. acarbose), insulins, meglitinides (e.g. repaglinide), sulfonylureas (e.g. glimepiride, glyburide and glipizide), biguanide/glyburide combinations (i.e., glucovance), thiozolidinediones (e.g. troglitazone, rosiglitazone and pioglitazone), PPAR-gamma agonists, aP2 inhibitors, and DP4 inhibitors; thyroid mimetics (including thyroid receptor antagonists) (e.g., thyrotropin, polythyroid, KB-130015, and dronedarone); Mineralocorticoid receptor antagonists such as spironolactone and eplerinone; growth hormone secretagogues; anti-osteoporosis agents (e.g., alendronate and raloxifene); hormone replacement therapy agents such as estrogen (including conjugated estrogens in premarin), and estradiol; antidepressants such as nefazodone and sertraline; antianxiety agents such as diazepam, lorazepam, buspirone, and hydroxyzine pamoate; oral contraceptives; anti-ulcer and gastroesophageal reflux disease agents such as famotidine, ranitidine, and omeprazole; anti-obesity agents such as orlistat; cardiac glycosides including digitalis and ouabain; phosphodiesterase inibitors including PDE III inhibitors (e.g. cilostazol), and PDE V inhibitors (e.g., sildenafil); protein tyrosine kinase inhibitors; steroidal anti-inflammatory agents such as prednisone, and dexamethasone; and other anti-inflammatory agents such as enbrel.

The above other therapeutic agents, when employed in combination with the compounds of the present invention, may be used, for example, in those amounts indicated in the Physicians' Desk Reference (PDR) or as otherwise determined by one of ordinary skill in the art.

Assays to determine the degree of activity of a compound as an IKur inhibitor are well known in the art and are described in references such as J. Gen. Physiol. April; 101(4):513-43, and Br. J. Pharmacol. 1995 May; 115(2):267-74.

Assays to determine the degree of activity of a compound as an inhibitor of other members of the Kᵥ1 subfamily are also well known in the art. For example, inhibition of Kᵥ1.1, Kᵥ1.2 and Kᵥ1.3 can be measured using procedures described by Grissmer S., et al., Mol. Pharmacol. 1994 June; 45(6):1227-34. Inhibition of Kᵥ1.4 can be measured using procedures described by Petersen K. R., and Nerbonne J. M., Pηlugers Arch. 1999 February; 437(3):381-92. Inhibition of Kᵥ1.6 can be measured using procedures described by Bowlby M. R., and Levitan I. B., J. Neurophysiol. 1995 June; 73(6):2221-9. And inhibition of Kᵥ1.7 can be measured using procedures described by Kalman K., et al., J. Biol. Chem. 1998 Mar. 6; 273(10):5851-7.

Compounds within the scope of the present invention demonstrate activity in Kᵥ1 assays such as the ones described above.

All documents cited in the present specification are incorporated herein by reference in their entirety.

The following examples and preparations describe the manner and process of making and using the invention and are illustrative rather than limiting. It is to be understood that there may be other embodiments which fall within the spirit and scope of the invention as defined by the claims appended hereto. Abbreviations employed herein are defined below.
abs.=absolute
KF=Karl Fischer
AP=area percent
Et=ethyl
h=hours
HPLC=high performance liquid chromatography
min=minutes
Bu=butyl
LOD=loss on drying
RT=room temperature
TFA=trifluoroacetic acid
g=gram

### EXAMPLES

The following Examples represent preferred embodiments of the invention.

### Example 1

### Preparation of H2-1 Dihydrate and H2-2 Dihydrate Crystalline Forms of (2S)-1-[[(7R)-7-(3,4-dichlorophenyl)-4,7-dihydro-5-methylpyrazolo[1,5]pyrimidine-6-yl]carbonyl]-2-(4-fluorophenyl)pyrrolidine

A reaction mixture formed of cold (0-5° C.) crude IKur compound (amorphous, H2-1 dihydrate and/or H2-2 dihyrate) (27.5 g, ˜55 mmol) in nBuOH (90 mL) and concentrated HCl (30 mL) was diluted with EtOH (40 mL), polish filtered and rinsed with EtOH (20 mL). The combined filtrate was transferred to a 3 necked 1 L round bottom flask equipped with mechanical stirrer. The filtrate flask was rinsed with EtOH (20 mL), and then the rinsing was combined with the filtrate. The solution (˜200 mL) was cooled to 4° C. NaOH (3 N, 80 mL) was added dropwise in 0.5 h so that the temperature was <8° C. HPLC indicated no decomposition. Saturated aqueous Na₃PO₄ (80 mL) was added followed by the addition of NaOH (3N, 8 mL) to adjust pH to 8.5. Precipitation of H2-1 dihydrate occurred immediately.

The reaction mixture was seeded with H2-2 crystalline form of the IKur compound (0.3 g, 0.6 mmol). The pH was adjusted to 7.5 with HCl (1N, 1 mL). The mixture was heated to 40° C. Form transformation (from H2-1 to H2-2) was monitored by XRD and was completed in 2 h at 40° C. pH was adjusted to 6.5-7.5 during the form transformation by adding ˜40 mL saturated Na₃PO₄ aqueous solution. The mixture was cooled to RT and stirred at RT for 2 h. The solid was collected by filtration. The cake was washed with EtOH:H₂O (1:1, 100 mL×2) and H₂O (100 mL×2). It was suction dried to a constant weight of 19 g, H2-2 crystalline form of the IKur compound, AP 99.4 and KF 6.9.

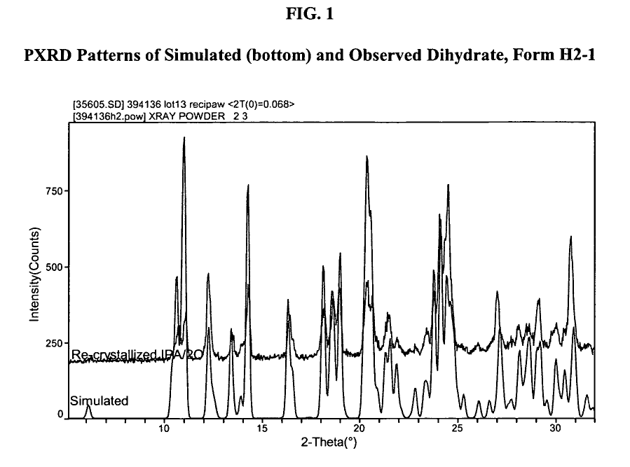

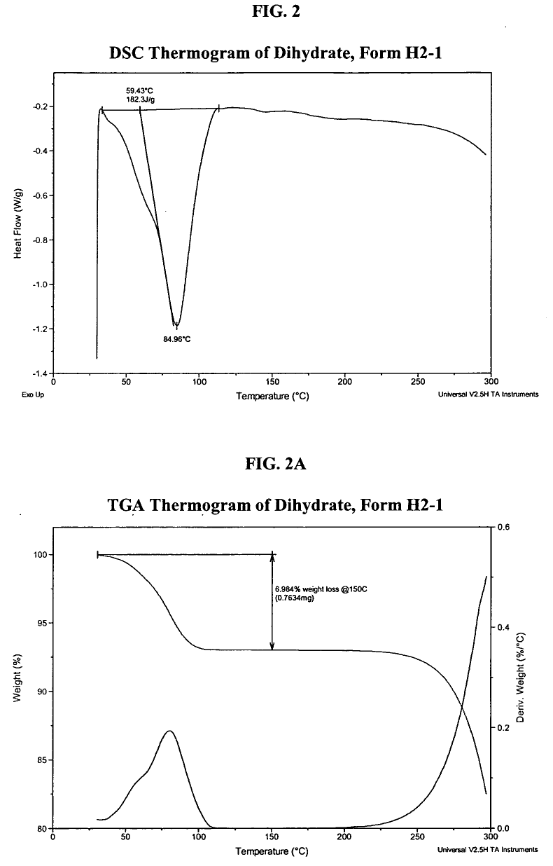

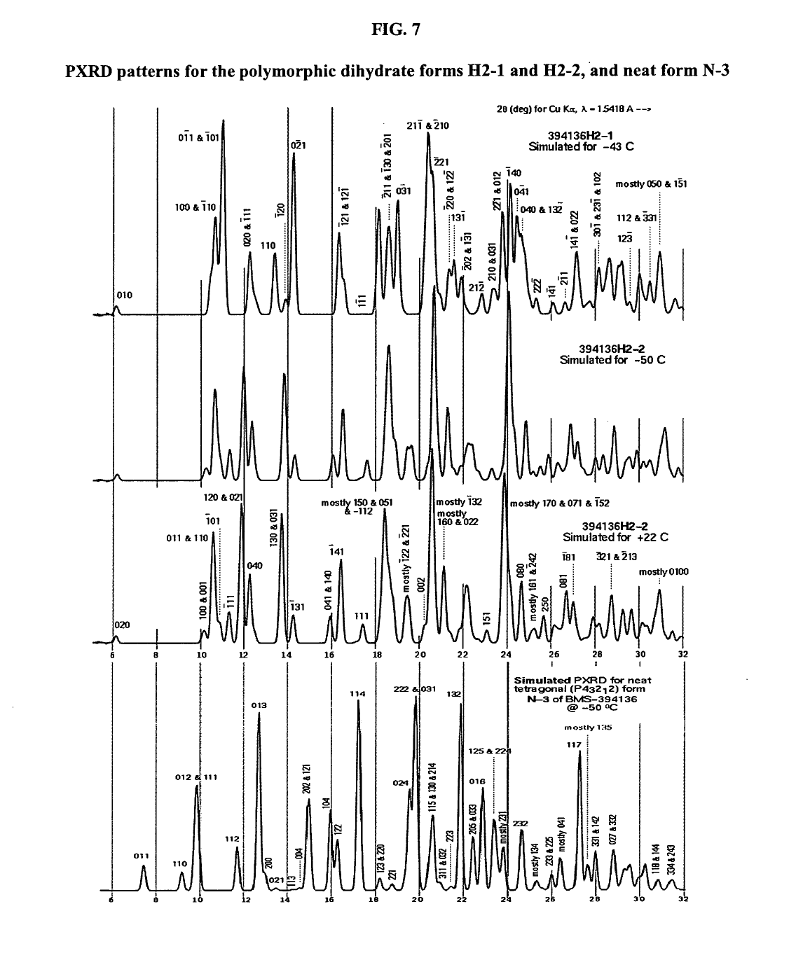

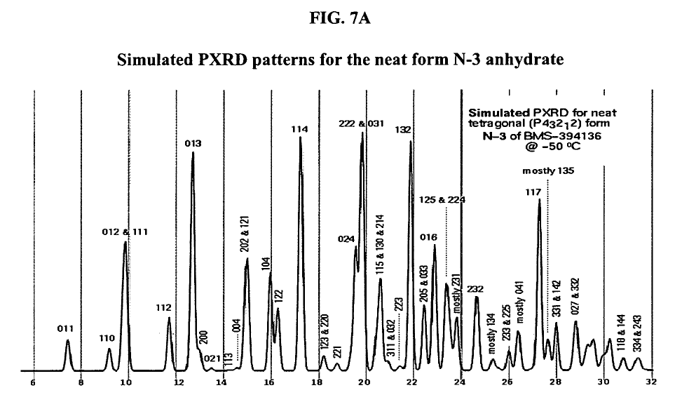

Observed and simulated powder x-ray diffraction (PXRD) patterns of the Example 1 H2-1 dihydrate and are shown in FIGS. 1 and 7, and differential scanning calimetry and thermogravimetric analysis thermograms thereof are shown in FIGS. 2 and 2A. In the observed PXRD pattern shown in FIG. 1, the first 5 strongest peaks are at 12.67 (±0.1)°, 14.18 (±0.1)°, 23.99 (±0.1)°, 24.42 (±0.1)°, and 30.70 (±0.1)° two theta.

Unit Cell Crystallographic Data for H2-1

| | | | | | | | | | | | | | | | Sol. |
|---|---|---|---|---|---|---|---|---|---|---|---|---|---|---|---|
| | | | | | | | | | | | | | | | Sites |
| Form | T | a(Å) | b(Å) | c(Å) | α° | β° | γ° | V(Å³) | Z′ | Vm | sg | dcalc | mp(° C.) | R | for Z′ |
| H2-1 | −43 | 9.760(2) | 15.119(4) | 9.439(3) | 97.82(2) | 115.69(2) | 99.88(2) | 1201(1) | 2 | 600 | P1 | 1.403 | (t)85, | .08 | 4H2O |
| | | | | | | | | | | | | | 108-113 | | |
| | | | | | | | | | | | | | (glass), | | |
| | | | | | | | | | | | | | 130-143° | | |

T = temp(° C.) for the crystallographic data
Z′ = number of drug molecules per asymmetric unit
Vm = V(unit cell)/(Z drug molecules per cell)

Single Crystal X-Ray Diffraction Measurement: H2-1 Dihydrate

| Refcode: 394136H2 | Crystal Form: | |
|---|---|---|
| BMS: 394136 | H2-1 | Accession No.: 1547 |
| X-ray Notebook No.: 46521-140 | | Chemist Notebook: 47655-173-23 |
| Chemical formula: C₂₄H₂₁N₄OFCl₂.2H₂O | | Crystallization solvent: MeCN/water |
| a: 9.760(2) Å | α: 97.82(2)° | Crystal description: colorless prisms |
| b: 15.119(4) Å | β: 115.69(2)° | Melting point: (t)85, (glass)108-113, 130-143° C. |
| c: 9.439(3) Å | γ: 99.88(2)° | Measured indices: ±h, ±k, ±l |
| V: 1201(1) Å³ | Z: 2 | Temperature (° C.): −43 |
| Space group: P1 | V/Z: 600 Å³ | (2θ)max, °: 130 |
| D_{calc} (g-cm⁻³): 1.403 | | No. of independent reflections: 5592 |
| Absorption coefficient, cm⁻¹: 28.2 | | No. of observed reflections (I ≧ 3σ): 5393 |
| Molecular volume (Vₘ): 832 | | No. refined variables: 621 |
| Molecular Surface Area: 880 | | R: 0.082 Renan: 0.092 |
| Packing coefficient (Z · Vₘ/V_{c}): 0.69 | | Rw: 0.10 Rw enan: 0.12 |
| Disorder: Dichlorophenyl disordered | | Avg. errors (C, N, O): 0.01 Å 0.6° |
| (4:1) | | |
| | | Solvent: 2 water sites Occupancy: 1.0 |
| | | chlathrate |

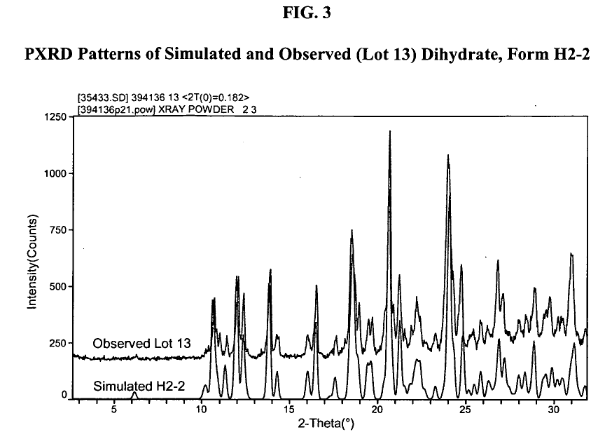

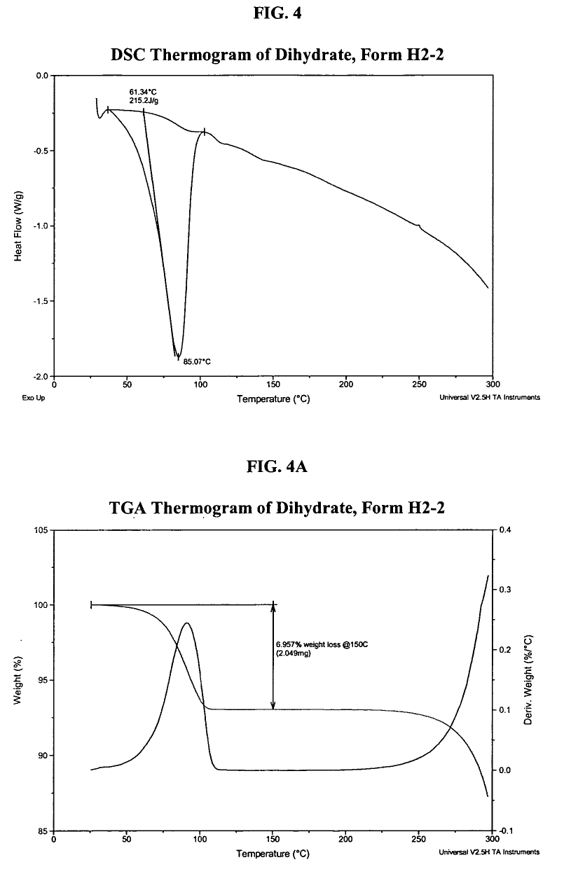

Observed and simulated powder x-ray diffraction patterns of the Example H2-2 dihydrate are shown in FIGS. 3 and 7, and differential scanning calimetry and thermogravimetric analysis thermograms of the Example 1 H2-2 dihydrate are shown in FIGS. 4 and 4A. In the observed PXRD pattern shown in FIG. 3, the first 5 strongest peaks are at 13.72 (±0.1)°, 18.35 (±0.1)°, 20.48 (±0.1)°, 23.78 (±0.1)°, and 26.63 (+±0.1)° two theta.

Powder X-Ray Diffraction Method Employed in All Examples:

About 200 mg sample was put into Philips PXRD sample holder by back loading method. Sample was radiated by Cu Kα₁, x-ray beam. Scanning conditions: continuous scanning mode at RT, 2 theta range: 2 to 32 degree, scanning rate: 0.03 degree/sec, auto divergence and anti scatter slits, receiving slit: 0.2 mm, sample spinner: ON. Instrument-Philips MPD Unit, power-45KV×40 mA.

Single Crystal X-ray Diffraction Measurement H2-2 Dihydrate

| Refcode: 394136HB | | |
|---|---|---|
| BMS: 394136 | Crystal Form: H2-2 | Accession No.: 1646 |
| X-ray Notebook No.: 50572-004 | | Chemist Notebook: lot 13 |
| Chemical formula: C₂₄H₂₁N₄OFCl₂.2H₂O | | Crystallization solvent: EtOH/H₂O/L-Pro |
| a: 9.7085(3) Å | α: 90° | Crystal description: tiny twinned colorless |
| | | prisms |
| b: 28.913(1) Å | β: 122.355(2)° | Melting point: (glass)105-107, 133-140° C. |
| c: 10.2911 Å | γ: 90° | Measured indices: +h, +k, ±l |
| V: 2440.2(1) Å3 | Z: 4 | Temperature (° C.): 25 |
| Space group: P21 | V/Z: 610 Å3 | (2θ max, °: 81 |
| Dcalc (g-cm−3): 1.381 | | No. of independent reflections: 2520 |
| Absorption coefficient, cm−1: 27.8 | | No. of observed reflections (I > 3σ): 2344 |
| Molecular volume (Vm): 840 | | No. refined variables: 613 |
| Molecular Surface Area: 977 | | R: 0.042 Renan: 0.053 |
| Packing coefficient (Z · Vm/Vc): 0.69 | | Rw: 0.051 Rw enan: 0.067 |
| Disorder: | | Avg. errors (C, N, O): 0.02 Å 1.5° |
| | | Solvent: 2 water sites Occupancy: 1.0 |
| | | clathrate |

Unit Cell Crystallographic Data for H2-2

| | | | | | | | | | | | | | | | Sol. Sites |
|---|---|---|---|---|---|---|---|---|---|---|---|---|---|---|---|
| Form | T | a(Å) | b(Å) | c(Å) | α° | β° | γ° | V(Å³) | Z′ | Vm | sg | dcalc | mp(° C.) | R | for Z′ |
| H2-2 | −50 | 9.674(1) | 28.690(1) | 10.248(2) | — | 122.72(1) | — | 2393.1(4) | 2 | 598 | P2₁ | 1.408 | 106(glass) | .06 | 4H2O |
| | | | | | | | | | | | | | 133-140 | | |
| H2-2 | 25 | 9.7085(3) | 28.913(1) | 10.353(2) | — | 122.89(2) | — | 2440.2(1) | 2 | 610 | P2₁ | 1.381 | | .04 | 4H20 |

T = temp(° C.) for the crystallographic data
Z′ = number of drug molecules per asymmetric unit
Vm = V(unit cell)/(Z drug molecules per cell)

Referring to FIG. 7, the simulated patterns were calculated from the refined atomic parameters; for H2-1 (top pattern), all four rotamers in the disordered crystal structure were included in the simulations. The intensity scales are somewhat arbitrary; ideally, for pure samples containing equal numbers of drug molecules, the peak height at 2θ˜16.3° in triclinic H2-1 (P1, Z=2) should be ˜80% of the peak height at 2θ˜16.4° in H2-2 (monoclinic, P21, Z=4).

### Example 2

### Form Transformation of IKur Compound from H2-2 Dihydrate Crystalline Form to N-3 Anydrate Crystalline Form and Particle Size Reduction

Materials:

| Compound | mmole |
|---|---|
| 1. IKur compound in form of H2-2 dihydrate (25.0 g) | 49.3 (1.00 eq.) |
| 2. Absolute EtOH (38 mL) | |

Procedure:

A 100 mL 3 necked round bottom flask was charged with IKur compound in the form of the H2-2 dihydrate (25.0 g). EtOH (38 mL) was added with stirring following by addition of seed (0.25 g IKur compound in the form of the N-3 anhydrate). The resulting slurry was heated to 40° C. for 10 h then at RT for 2 days under a blanket of nitrogen. It was cooled to 0-5° C. in 15 minutes and stirred at 0-5° C. for 1 h. The mixture was filtered and rinsed with the mother liquor. The cake was suction dried for 1 h at RT. It was then transferred to a crystallization dish. The material was dried in a vacuum oven at 50° C. for 2 h then at RT for 13 h at 27 mmHg to give 22.4 g of white crystalline solid in 92% yield, AP 99.5.

### Analytical Data

Gradient HPLC Method:

| Instrument: | Shimadzu HPLC | | | | |
|---|---|---|---|---|---|
| Column: | Phenomenex Luna 3 μm, 4.6 mm i.d. × 150 mm, | | | | |
| | C8(2) | | | | |
| Temperature: | 25° C. | | | | |
| Mobile Phase: | A = CH₃CN:TFA (0.1%) | | | | |
| | B = Water:TFA (0.1%) | | | | |
| Gradient: | Step | Time | A | B | |
| | 0 | 0.0 | 49 | 60 | |
| | 1 | 25.0 | 80 | 20 | |
| | 2 | 1.0 | 40 | 60 | |
| | 3 | 9.0 | 40 | 60 | linear gradient |

| | |
|---|---|
| Flow Rate: | 1.0 mL/min |
| Detection: | UV absorption at 238 nm |
| Injection Volume: | 15 μL |
| Run Time: | 30 min |

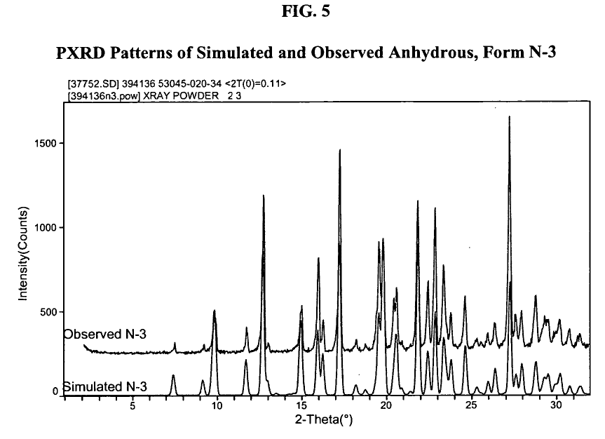

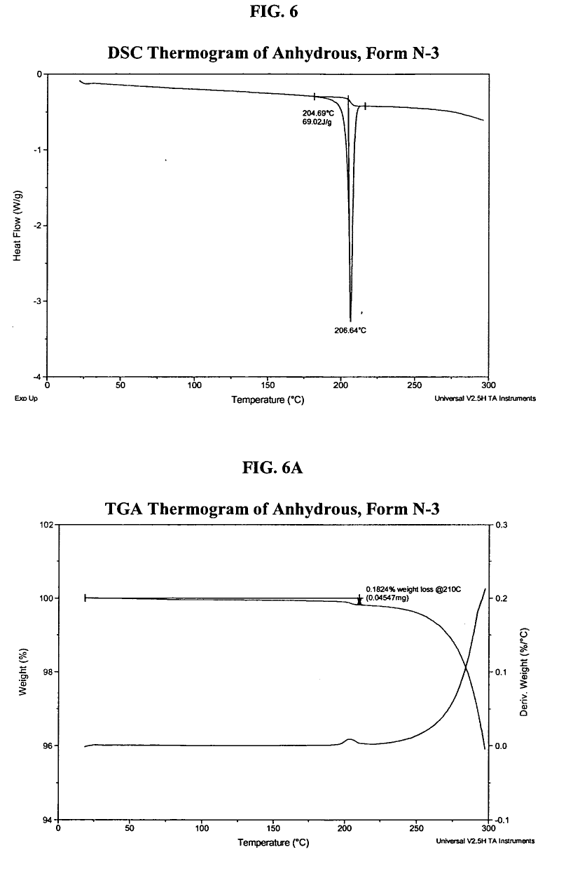

Observed and simulated powder x-ray diffraction patterns of the Example H2-2 dihydrate are shown in FIGS. 5, 7 and 7A, and differential scanning calimetry and thermogravimetric analysis thermograms of the Example 2 N-3 anhydrate are shown in FIGS. 6 and 6A. In the observed PXRD pattern shown in FIG. 5, the first 5 strongest peaks are at 12.63 (±0.1)°, 17.14 (±0.1)°, 21.74 (±0.1)°, 22.76 (±0.1)°, and 27.11 (+0.1)° two theta.

Unit Cell Crystallographic Data for N-3 Anhydrate

| | | | | | | | | | | | | | | | Sol. Sites |
|---|---|---|---|---|---|---|---|---|---|---|---|---|---|---|---|
| Form | T | a(Å) | b(Å) | c(Å) | α° | β° | γ° | V(Å³) | Z′ | Vm | sg | dcalc | mp(° C.) | R | for Z′ |
| N-3 | −50 | 13.6528(1) | 13.6528(1) | 24.364(1) | — | — | — | 4541.5(1) | 1 | 568 | P4₃2₁2 | 1.379 | 212-213 | .04 | none |

T = temp(° C.) for the crystallographic data
Z′ = number of drug molecules per asymmetric unit
Vm = V(unit cell)/(Z drug molecules per cell)

### Example 3

### Preparation of MTBE Solvate (P-1)

500 mg of the IKur compound (in the form of H2-1 dihydrate crystals) was slurried in 5 mL methyl t-butyl ether (MTBE) at room temperature.

The resulting slurry was seeded with MTBE solvate (0.005 gm).

After about 1 week, the H2-1 dihydrate form converted to the MTBE solvate form which was chemically stable.

HPLC, purity ˜98.2%.

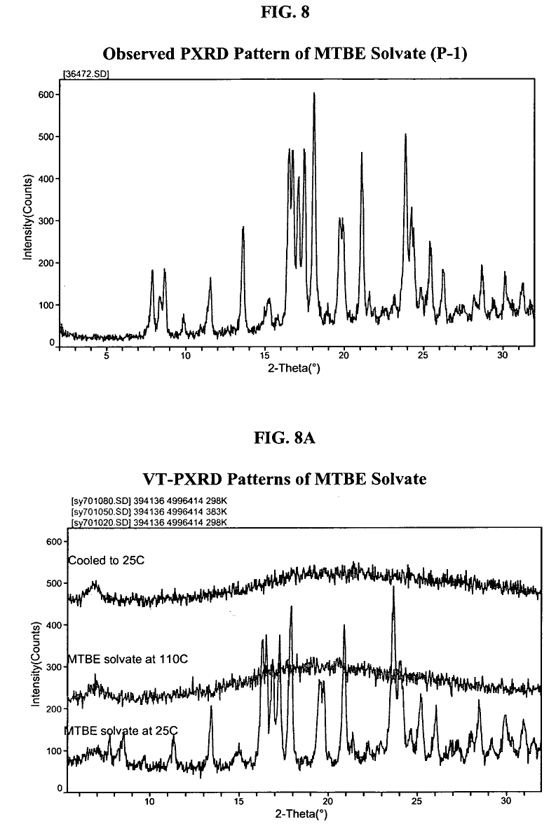

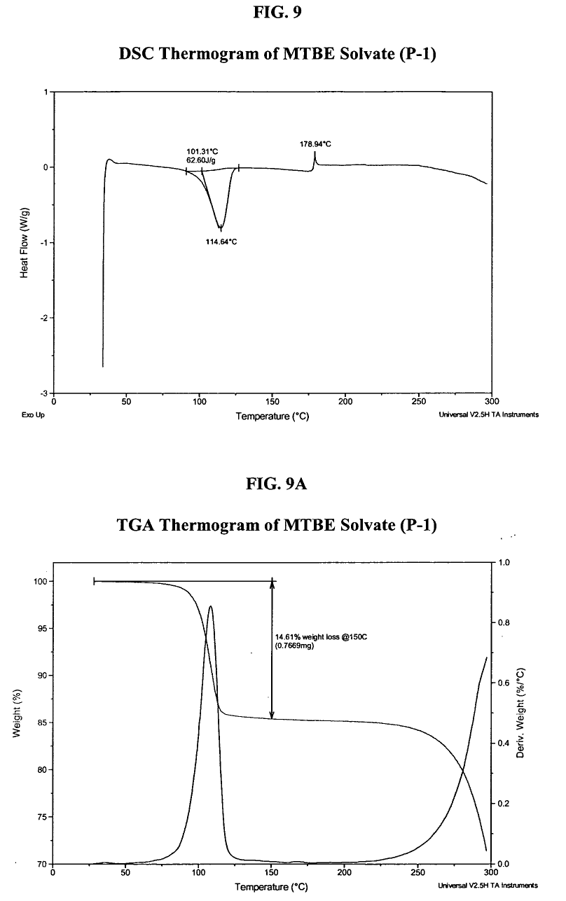

Observed PXRD and VT-PXRD, and differential calimetry and thermogravimetric analysis thermograms of the Example 3 MTBE solvate obtained are shown in FIGS. 8, 8A and 9 and 9A, respectively. In FIG. 8, the first 5 strongest peaks in the PXRD are at: 16.51 (±0.1)°, 17.11 (±0.1)°, 18.08 (±0.1)°, 21.10 (±0.1)°, and 23.87 (±0.1)° two theta.

### Example 4

### Preparation of H2-1 Dihydrate and H2-2 Dihydrate

A solution of 300 ml n-butyl alcohol, 100 mL 12N HCl and 90 g IKur compound (in the form of a mixture of amorphous, H2-1 dihydrate and H2-2 dihydrate forms) was prepared. 460 mL abs. ethanol was added to the above solution containing the IKur compound. The reaction mixture was polish-filtered (filter paper Whatman #4) and then transferred to a 2-L 3-neck indented round bottom flask and maintained at 0-5° C.

3N NaOH (330 mL) was charged to the reaction mixture in 15-20 min while maintaining the batch temperature <10° C.

The resulting solution became cloudy initially due to the precipitation of NaCl. After ˜200 mL 3N NaOH was added, the solution became significantly clearer. The H2-1 dihydrate started to precipitate after 300 mL NaOH was added. pH of the slurry was ca. 1-2 after NaOH addition was completed.

1 gm seeds of H2-2 dihydrate was added to the slurry. ˜115 mL saturated Na₃PO₄ aqueous solution was charged to the seeded reaction mixture to adjust pH of the mixture to 6.5-8.5. Additional saturated Na₃PO₄ (40 mL) was added to the reaction mixture to maintain pH within 6 to 7.5. The resulting slurry was heated to 40° C.

Periodically x-ray diffraction samples were taken to monitor the transformation from H2-1 dihydrate to H2-2 dihydrate.

After 1 to 3 hours, pure H2-2 dihydrate was obtained. 115 mL deionized water was added to the slurry. The slurry was cooled to 2° C. over 1-2 hours. The slurry was held at 2° C. for at least ½ hour and then was filtered using a Buchner funnel and filter paper (Whatman #4). The wet cake was washed with 300 mL (˜3 cake volume) ethanol/water (1/2) followed by an H₂O wash until conductivity of the filtrate was ca. 0.001Ω⁻¹.

The cake was suction dried with mild house vacuum. The cake was stirred periodically for homogeneity. Drying was continued until LOD (by TGA) of the wet cake was ca. 7.1%. 67 g of H2-2 dihydrate was obtained with in process HPLC AP=99%, particle size D95=75 μm. The drying process was also monitored by in-line near IR.

Observed and simulated powder x-ray diffraction patterns, and differential scanning calimetry and thermogravimetric analysis thermograms of the H2-2 dihydrate crystals produced in Example 4 are shown in FIGS. 3, 7, and 4 and 4A, respectively.

### Example 5

### Recrystallization to Prepare H2-2 Dihydrate

128 g IKur compound (a mixture of amorphous, H2-1 dihydrate and H2-2 dihydrate forms) was dissolved in 560 mL absolute ethanol at 2.5° C. The resulting solution was polish filtered.

To a 2-L 3-neck indented round bottom flask were added 560 mL deionized water and 0.3 g H2-2 dihydrate seeds. The resulting slurry was stirred at 25° C.

The rich solution (˜600 mL) was added to the thin H₂O slurry (containing the H2-2 dihydrate seeds) through a dropping funnel over 1-1.5 hours.

The resulting slurry was heated to 50° C. 280 mL absolute ethanol was added to the heated slurry over 20 min. 1 g H2-2 dihydrate seeds was added upon completion of the ethanol addition.

XRD samples were periodically monitored for transformation of H2-1 dihydrate to H2-2 dihydrate.

The slurry was cooled to 2° C. over 2 hours after pure H2-2 dihydrate was obtained (4 to 10 hours). The reaction slurry was held at 2° C. for at least ½ hour before filtration. The cold slurry was filtered employing a Buchner funnel and filter paper (Whatman #4). The cake was washed with ˜2 cake volume of ethanol/water (1/2) and ˜4 cake volume of water. The resulting wet cake was suction dried. 118 g of H2-2 dihydrate was obtained (yield=92.2%), particle size D95=25 μm.

Observed and simulated powder x-ray diffraction patterns, and thermogravimetric analysis thermograms of the H2-2 dihydrate crystals produced in Example 5 are shown in FIGS. 3, 7, and 4 and 4A, respectively.

### Example 6

### Oral Solution of N-3 Anhydrate

An oral solution of the following composition was prepared as described below.

| Ingredients (Vehicle) | Amount (% w/w) | |
|---|---|---|
| d-alpha tocopheryl polyethylene glycol 1000 | 20 | |
| succinate (TPGS) | | |
| polyoxyethylene (20) sorbitan monooleate (Tween 80 | 8.5 | |
| or Polysorbate 80) | | |
| 95% ethanol, USP | 10 | |
| polyethylene glycol 400 (PEG 400) | 61.5 | |
| N-3 anhydrate | 30 | mg/mL |

The vehicle was prepared by first dissolving the TPGS in ethanol and then adding the appropriate amounts of PEG 400 and Polysorbate 80.

The vehicle was then mixed with the N-3 anhydrate to form the oral solution.

### Example 7

### Oral Suspension of N-3 Anhydrate

An oral suspension of the following composition was prepared as described below.

| N-3 anhydrate | 2 to 10 | mg/mL |
|---|---|---|
| Polysorbate 80 | 0.05% | w/w |
| Purified Water | 99.95% | w/w |

The oral suspension was prepared by mixing the Polysorbate 80 and water and then mixing with the N-3 anhydrate.

What is claimed is:

1. a) The H2-1 dihydrate crystalline form of the free base of the structure 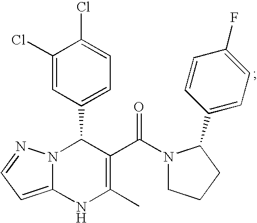
b) the H2-2 dihydrate crystalline form of the free base of the structure as defined above;
c) the N-3 anhydrate crystalline form of the free base of the structure as defined above; or
d) the MTBE solvate of the free base of the solvate as defined above.

2. The H2-1 dihydrate crystalline form of the free base of the structure 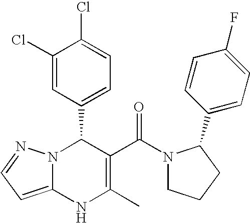
as defined in claim 1, as characterized by the simulated and observed powder x-ray diffraction patterns shown in FIG. 1 or in the observed pattern, by the first 5 major peaks at 12.67 (±0.1)°, 14.18 (±0.1)°, 23.99 (±0.1)°, 24.42 (±0.1)°, and 30.70 (±0.1)° two theta, as shown in FIG. 1, and by the thermograms shown in FIGS. 2 and 2A.
Single Crystal X-Ray Diffraction Measurement: H2-1 Dihydrate

| Refcode: 394136H2 | Crystal Form: | |
|---|---|---|
| BMS: 394136 | H2-1 | Accession No.: 1547 |
| X-ray Notebook No.: 46521-140 | | Chemist Notebook: 47655-173-23 |
| Chemical formula: C₂₄H₂₁N₄OFCl₂.2H₂O | | Crystallization solvent: MeCN/water |
| a: 9.760(2) Å | α: 97.82(2)° | Crystal description: colorless prisms |
| b: 15.119(4) Å | β: 115.69(2)° | Melting point: (t)85, (glass)108-113, 130-143° C. |
| c: 9.439(3) Å | γ: 99.88(2)° | Measured indices: ±h, ±k, ±l |
| V: 1201(1) Å³ | Z: 2 | Temperature (° C.): −43 |
| Space group: P1 | V/Z: 600 Å³ | (2θ)max, °: 130 |
| D_{calc} (g-cm⁻³): 1.403 | | No. of independent reflections: 5592 |
| Absorption coefficient, cm⁻¹: 28.2 | | No. of observed reflections (I ≧ 3σ): 5393 |
| Molecular volume (Vₘ): 832 | | No. refined variables: 621 |
| Molecular Surface Area: 880 | | R: 0.082 Renan: 0.092 |
| Packing coefficient (Z · Vₘ/V_{c}): 0.69 | | Rw: 0.10 Rw enan: 0.12 |
| Disorder: Dichlorophenyl disordered | | Avg. errors (C, N, O): 0.01 Å 0.6° |
| (4:1) | | |
| | | Solvent: 2 water sites Occupancy: 1.0 |
| | | chlathrate |

3. The H2-2 dihydrate crystalline form of the free base of the structure 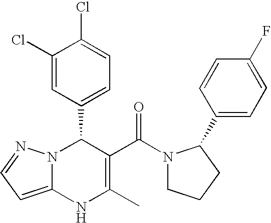
as defined in claim 1, as characterized by the simulated and observed powder x-ray diffraction patterns or in the observed pattern, by the first 5 major peaks: 13.72 (±0.1)°, 18.35 (±0.1)°, 20.48 (±0.1)°, 23.78 (±0.1)°, and 26.63 (±0.1)° two theta, as shown in FIG. 3, and by the thermograms shown in FIGS. 4 and 4A.
Single Crystal X-ray Diffraction Measurement H2-2 Dihydrate

| Refcode: 394136HB | | |
|---|---|---|
| BMS: 394136 | Crystal Form: H2-2 | Accession No.: 1646 |
| X-ray Notebook No.: 50572-004 | | Chemist Notebook |
| Chemical formula: C₂₄H₂₁N₄OFCl₂.2H₂O | | Crystallization solvent: EtOH/H₂O/L-Pro |
| a: 9.7085(3) Å | α: 90° | Crystal description: tiny twinned colorless |
| | | prisms |
| b: 28.913(1) Å | β: 122.355(2)° | Melting point: (glass)105-107, 133-140° C. |
| c: 10.2911 Å | γ: 90° | Measured indices: +h, +k, ±l |
| V: 2440.2(1) Å3 | Z: 4 | Temperature (° C.): 25 |
| Space group: P21 | V/Z: 610 Å3 | (2θ.max, °: 81 |
| Dcalc (g-cm−3): 1.381 | | No. of independent reflections: 2520 |
| Absorption coefficient, cm−1: 27.8 | | No. of observed reflections (I > 3σ): 2344 |
| Molecular volume (Vm): 840 | | No. refined variables: 613 |
| Molecular Surface Area: 977 | | R: 0.042 Renan: 0.053 |
| Packing coefficient (Z · Vm/Vc): 0.69 | | Rw: 0.051 Rw enan: 0.067 |
| Disorder: | | Avg. errors (C, N, O): 0.02 Å 1.5° |
| | | Solvent: 2 water sites Occupancy: 1.0 |
| | | clathrate |

4. The N-3 anhydrate crystalline form of the free base of the structure 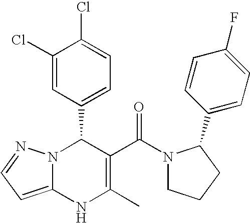
as defined in claim 1, as characterized by the simulated and observed powder x-ray diffraction patterns, or in the observed pattern, by the first 5 major peaks: 12.63 (±0.1)°, 17.14 (±0.1)°, 21.74 (±0.1)°, 22.76 (±0.1)°, and 27.11 (±0.1)° two theta, as shown in FIG. 5, and by the thermograms shown in FIGS. 6 and 6A.

5. The MTBE solvate crystalline form of the free base of the structure 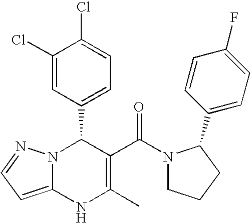
as defined in claim 1, as characterized by the observed powder x-ray diffraction patterns or the first 5 major peaks: 16.51 (±0.1)°, 17.11 (±0.1)°, 18.08 (±0.1)°, 21.10 (±0.1)°, and 23.87 (±0.1)° two theta, as shown in FIGS. 8 and 8A, and by the thermograms shown in FIGS. 9 and 9A.

6. A process for preparing the N-3 anhydrate crystalline form of the IKur compound of the structure 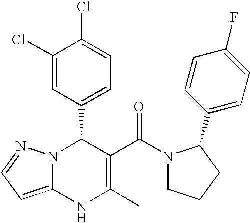
as defined in claim 4, which comprises:
a) providing the H2-2 dihydrate crystalline form of the IKur compound;
b) treating H2-2 dihydrate crystalline form with ethyl alcohol;
c) optionally seeding the reaction mixture from step b) with N-3 anhydrate crystalline form of the IKur compound to form a reaction slurry;
d) heating the slurry from step c) at an elevated temperature up to about 50° C.; and
e) drying the resulting product and recovering N-3 anhydrate crystals of the IKur compound.

7. The process as defined in claim 6 wherein the reaction mixture from step b) is treated with seeds of N-3 anhydrate crystals of the IKur compound.

8. The process as defined in claim 6 wherein the reaction mixture from step b) or step c) is heated at a temperature within the range from about 35 to about 50° C. for a period within the range from about 0.5 to about 20 hours, and the reaction mixture is then maintained at ambient temperature for a period from about 0.5 hours to about 3 days under an inert atmosphere.

9. The process as defined in claim 6 wherein the ethyl alcohol is employed in a molar ratio to the H2-2 dihydrate within the range from about 14:1 to about 42:1, and the seeds of the N-3 anhydrate crystalline form are employed in a molar ratio to the H2-2 dihydrate crystalline form within the range from about 0.001:1 to about 0.020:1.

10. A process for preparing the H2-1 dihydrate crystalline form of the IKur compound of the structure 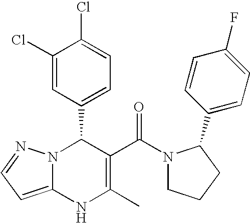
as defined in claim 2, which comprises:
a) providing the IKur compound in the form of an amorphous solid, H2-1 dihydrate or H2-2 dihydrate and/or a mixture of two or more thereof;
b) optionally forming a mixture of the IKur compound in n-butyl alcohol, and concentrated hydrochloric acid;
c) mixing the IKur compound from step a) or step b) with ethyl alcohol at a reduced temperature below about 5° C.; and
d) treating the reaction mixture from step b) or step c) with a strong base while maintaining the reaction mixture at a temperature within the range from about 0° C. to about −25° C., and optionally adding trisodium phosphate to adjust pH of the reaction mixture within the range from about 6.5 to about 9, to cause precipitation of the H2-1 dihydrate.

11. The process as defined in claim 10 wherein the butyl alcohol is employed in a molar ratio to the starting IKur compound within the range from about 12:1 to about 24:1, and the concentrated hydrochloric acid is employed in a molar ratio to the starting IKur compound within the range from about 4:1 to about 10:1.

12. The process as defined in claim 10 wherein the reaction mixture from step a) is treated with ethyl alcohol employing a molar ratio of ethyl alcohol to IKur compound within the range from about 30:1 to about 60:1.

13. The process as defined in claim 10 wherein the reaction mixture from step b) or step c) is treated with NaOH and trisodium phosphate to adjust pH of the reaction mixture within the range from about 6 to about 7.5.

14. A process for preparing the H2-2 dihydrate crystalline form of the IKur compound of the structure 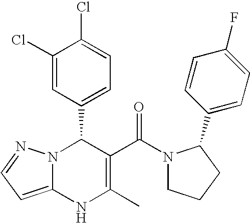
as defined in claim 3, which comprises:
a) providing an IKur compound in the form of an amorphous solid, H2-1 dihydrate, H2-2 dihydrate and/or a mixture of two or more thereof at a reduced temperature below about 2.5° C.;
b) mixing the IKur compound from step a) with ethyl alcohol;
c) treating the reaction mixture from step b) with a base and optionally trisodium phosphate to adjust pH within the range from about 6.5 to about 9 while maintaining the reaction mixture at a temperature within the range from about 0 to about −2.5° C., to cause precipitation of the H2-1 dihydrate of the IKur compound and form a reaction slurry;
d) seeding the reaction slurry containing H2-1 dihydrate with crystals of H2-2 dihydrate;
e) adjusting pH of the reaction mixture from step d) or the reaction slurry from step c) within the range from about 5.5 to about 8.5; and
f) heating the reaction mixture from step e) at a temperature within the range from about 30 to about 50° C. to form the H2-2 dihydrate.

15. The process as defined in claim 14 including the step of mixing ethyl alcohol with the reaction mixture of step a).

16. The process as defined in claim 14 including the step of seeding the reaction mixture from step c) with crystals of H2-2 dihydrate employing a molar ratio of H2-2 dihydrate: H2-1 dihydrate within the range from about 0.001:1 to about 0.020:1.

17. A process for preparing the methyl t-butyl ether (MTBE) solvate crystalline form of the IKur compound of the structure 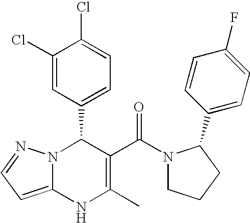
as defined in claim 5, which comprises:
a) forming a slurry of the IKur compound in MTBE; and
b) mixing the slurry from step a) with seeds of MTBE solvate to form MTBE solvate.

18. A process for preparing the H2-2 dihydrate crystalline form of the IKur compound 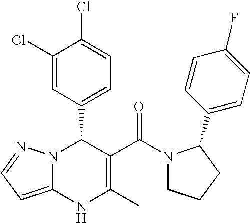
as defined in claim 3, which includes the steps of:
a) dissolving the IKur compound, in the form of an amorphous solid, H2-1 dihydrate, H2-2 dihydrate or a mixture of two or more thereof, in ethyl alcohol;
b) adding the solution of IKur compound to an aqueous slurry of seeds of H2-2 dihydrate crystalline form of the IKur compound to form a slurry;
c) heating the slurry from step b) at a temperature within the range from about 35° C. to about 65° C.;
d) optionally adding ethyl alcohol to the reaction mixture from step c);
e) optionally adding additional seeds of H2-2 dihydrate crystalline form to the reaction mixture from step c) or step d) to form H2-2 dihydrate;
f) optionally cooling the reaction mixture from step e) at a temperature within the range from about −5 to about 10° C.; and
g) recovering H2-2 dihydrate.

19. The process as defined in claim 18 wherein the seeds of H2-2 dihydrate are employed in a molar ratio to the starting IKur compound within the range from about 0.001:1 to about 0.010:1.

20. The process as defined in claim 18 wherein the reaction mixture from step c) is treated with ethyl alcohol employing a molar ratio of ethyl alcohol to IKur compound within the range from about 10:1 to about 30:1.

21. The process as defined in claim 18 including the step of mixing ethyl alcohol with the reaction mixture of step c).

22. The process as defined in claim 18 including the step of seeding the reaction mixture from step d) with crystals of H2-2 dihydrate employing a molar ratio of H2-2 dihydrate: H2-1 dihydrate within the range from about 0.001:1 to about 0.015:1.

23. The H2-1 dihydrate crystalline form of the free base of the structure 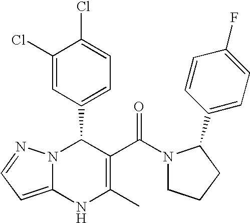
prepared by the process as defined in claim 10.

24. The H2-2 dihydrate crystalline form of the free base of the structure 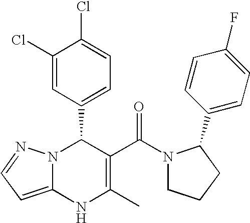
prepared by the process as defined in claim 14.

25. The N-3 anhydrate crystalline form of the free base of the structure 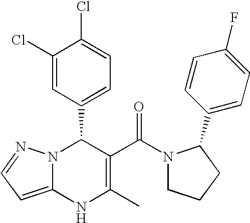
prepared by the process as defined in claim 6.

26. The MTBE solvate crystalline form of the free base of the structure 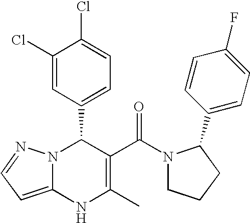
prepared by the process as defined in claim 17.

27. A pharmaceutical composition comprising at least one compound according to claim 1 and a pharmaceutically-acceptable carrier or diluent.

28. A pharmaceutical composition comprising at least one compound according to claim 4 and a pharmaceutically-acceptable carrier or diluent.

29. A method of treating arrhythmia disorder comprising administering to a patient in need of such treatment a pharmaceutical composition according to claim 1.

30. The method of claim 29 in which the inflammatory disorder is selected from asthma, adult respiratory distress syndrome, chronic obstructive pulmonary disease, chronic pulmonary inflammatory disease, diabetes, inflammatory bowel disease, osteoporosis, psoriasis, graft vs. host rejection, atherosclerosis, and arthritis including rheumatoid arthritis, psoriatic arthritis, traumatic arthritis, rubella arthritis, gouty arthritis and osteoarthritis.

31. The pharmaceutical composition as defined in claim 27 in the form of an oral solution comprising:
a) N-3 anhydrate, H2-1 dihydrate, H2-2 dihydrate, amorphous form or MTBE solvate;
b) d-alpha tocopheryl polyethylene glycol 1000 succinate (TPGS);
c) polyoxyethylene (20) sorbitan monooleate;
d) ethanol; and
e) polyethylene glycol 400.

32. The composition as defined in claim 31 comprising:
N-3 anhydrate—5 to 30 mg/mL, and a carrier comprising:
a) TPGS—15 to 20% w/w;
b) polyoxyethylene (20) sorbitan monooleate—7 to 10% w/w;
c) ethanol—8 to 12% w/w; and
d) polyethylene glycol 400—50 to 70% w/w.

33. The composition as defined in claim 32 having the following composition:
N-3 anhydrate—30 mg/mL and a carrier comprising:
a) TPGS—20% w/w;
b) polyoxyethylene (20) sorbitan monooleate—8.5% w/w;
c) ethanol—10% w/w; and
d) polyethylene glycol 400—61.5% w/w.

34. The pharmaceutical composition as defined in claim 27 in the form of an oral suspension comprising:
a) N-3 anhydrate, H2-1 dihydrate, H2-2 dihydrate, amorphous form or MTBE solvate;
b) polyoxyethylene (20) sorbitan monooleate; and
c) purified water.

35. The composition as defined in claim 34 comprising:
N-3 anhydrate—2 to 10 mg/mL, and a carrier comprising:
a) polyoxyethylene (20) sorbitan monooleate—0.01 to 1% w/w; and
b) purified water—98 to 99.95% w/w.

36. The composition as defined in claim 34 comprising:
N-3 anhydrate—2 mg/mL, and a carrier comprising:
a) polyoxyethylene (20) sorbitan monooleate—0.05% w/w; and
b) purified water—99.95% w/w.
